(19) United States
(12) Patent Application Publication
(10) Pub. No.: US 2010/0151497 A1
(43) Pub. Date: Jun. 17, 2010

(54) FLUORESCENCE-BASED ASSAY FOR DETECTING COMPOUNDS FOR MODULATING THE SODIUM-CALCIUM EXCHANGER (NCX) IN "FORWARD MODE"

(75) Inventors: Martin Hug, Freiberg (DE); Thomas Licher, Frankfurt am Main (DE); Sven Geibel, Frankfurt am Main (DE); Henning Vollert, Frankfurt am Main (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/529,230

(22) PCT Filed: Mar. 4, 2008
(86) PCT No.: PCT/EP08/01707

§ 371 Date: Mar. 3, 2010

(30) Foreign Application Priority Data
Mar. 13, 2007 (DE) 10 2007 011 913.7

(51) Int. Cl. G01N 33/567

(52) U.S. Cl. 435/7.21

(57) ABSTRACT

Transporters are an emerging target family with enormous potential, offering scientific and economic opportunities. The Sodium/Calcium exchanger is an important mechanism for removing Ca²⁺ from diverse cells. In heart, it extrudes Ca²⁺ that has entered through Ca²⁺ channels to initiate contraction, while Na⁺ enters the heart cell. It is of considerable interest to identify compounds that modulate the activity of Sodium/Calcium exchangers.

The present invention is directed to a fluorescence-based assay for detecting NCX “forward mode” modulating compounds. It further refers to a kit of parts comprising cells expriming NCX and the use of the kit of parts to test a compound for activity as an agonist or antagonist of NCX.

### FIELD OF THE INVENTION

The present invention relates to sodium-calcium exchangers (NCX) and methods for determining their activity. More specifically, the invention relates to a fluorescence-based assay for detecting NCX “forward mode” modulating compounds. It further refers to a kit of parts comprising cells expriming NCX and the use of the kit of parts.

### BACKGROUND OF THE INVENTION

A basic requirement for life is compartmentalization—with biological membranes being nature's tool to realize this principle. However, a lipid bilayer—the structure underlying the cell membrane—is impermeable to most ions and compounds whose transport is essential to sustain vital functions in cells and organisms. The answer to this paradox lies in the semi-permeable nature of the cell membrane—solutes that have to cross the membrane are transported by specific membrane proteins. These transporters are responsible for the generation and maintenance of ion gradients, the uptake of nutrients, the transport of metabolites, the reuptake of signaling molecules and the disposal of toxic and waste compounds. Therefore, transporters are potential drug targets that allow direct influence on disease-related abnormalities in this context.

The Sodium/Calcium exchanger is an important mechanism for removing Ca²⁺ from diverse cells. In heart, it extrudes Ca²⁺ that has entered through Ca²⁺ channels to initiate contraction, while Na⁺ enters the heart cell. Its relevance in cardiovascular diseases is e.g. illustrated in Hobai, J A & O'Rourke, B (2004) Expert Opin. Investig. Drugs, 13, 653-664. Therefore, pharmaceutical industry has developed compounds inhibiting the NCX as e.g. described in Iwamoto, T. et al. (2004) J. Biol. Chem., 279, 7544-7553. The Na⁺/Ca²⁺ exchanger electrogenically transports three to four Na⁺ for every Ca²⁺ that moves in the opposite direction as e.g. shown by electrophysiological means in Hinata, M. et al. (2002) J. Physiol. 545, 453-461. The NCX is able to maintain the cytoplasmic Ca²⁺ concentration ([Ca²⁺] in) three to four orders of magnitude below the extracellular Ca²⁺ concentration ([Ca²⁺] out). Nevertheless, the direction of net Ca²⁺ transport depends on the electrochemical gradient of Na⁺. Simultaneous and consecutive transport models have been suggested for Na⁺ and Ca²⁺ translocations, and a bulk of evidence favors the latter.

Transporters are an emerging target family with enormous potential, offering scientific and economic opportunities. On the other hand, transporters are a difficult target class in terms of drug-discovery technologies.

It is of considerable interest to identify compounds that modulate channel activity, for example, by blocking the flow of calcium and/or inhibiting the activation of calcium channels. One standard method to do so is through the use of patch clamp experiments.

In these experiments, cells must be evaluated individually and in sequence by highly skilled operators, by measuring the calcium current across the cell membrane in response to changes of the membrane potential and/or application of test compounds. The effect of Sea0400, a new specific inhibitor of NCX, on the action potential in dog ventricular papillary muscle was investigated and disclosed by K. Acsai during the “ESC Congress 2004” in Munich on Poster Nr. 2886 (Title: Effect of a specific sodium-calcium exchanger blocker Sea0400 on the ventricular action potential and triggered activity in dog ventricular muscle and Purkinje fiber) and by C. Lee et al. (The journal of pharmacology and experimental therapeutics; Vol. 311: 748-757, 2004; Title: Inhibitory profile of SEA0400 [2-[4-[(2,5-Difluorophenyl)methoxy]phenoxy]-5-ethoxyaniline] assessed on the cardiac Na⁺/Ca²⁺ exchanger, NCX1.1).

It was shown, using an ion-selective electrode technique to quantify ion fluxes in giant patches, that the cardiac Na⁺/Ca²⁺ exchanger has multiple transport modes (Tong Mook Kang & Donald W. Hilgemann; Nature; Vol. 427, 5 Feb. 2004; Title: Multiple transport modes of the cardiac Na⁺/Ca²⁺ exchanger).

These experiments, while valid and informative, are very time consuming and not adaptable to high-throughput assays for compounds that modulate calcium ion channel activity.

Various techniques have been developed as alternatives to standard methods of electrophysiology. For example, radioactive flux assays have been used in which cells are exposed with a radioactive tracer (e.g., ⁴⁵Ca) and the flux of the radio-labeled Ca is monitored. Cells loaded with the tracer are exposed to compounds and those compounds that either enhance or diminish the efflux of the tracer are identified as possible activators or inhibitors of ion channels in the cells' membranes. A specific example is enclosed in T. Kuramochi et al.; Bioorganic & Medicinal Chemistry; 12 (2004) 5039-5056; Title: Synthesis and structure-activity relationships of phenoxypyridine derivates as novel inhibitors of the sodium-calcium exchanger. EP1031556 discloses a method wherein Na⁺/Ca²⁺ exchanger activity is measured using sarcolemmal vesicles, the concentration of Ca²⁺ uptake in the sarcolemmal vesicles being determined by measuring ⁴⁵Ca radioactivity.

Many radioactive ion-transporter assays have limited sensitivity and therefore insufficient date quality. In addition, the cost and safety issues associated with the radioactive screening technology are hurdles that hinder a broadened application.

Among the above cited drug-discovery technologies, the use of radioactive flux assays to identify compounds that modulate the activity of ion channels and ion transporters is the closest prior art to our invention as it is a technique in which a test compound can be identified as possible activator or inhibitor by monitoring the flux of Ca²⁺ from the cells. The main issue for the radioactive assays is based on the difficulty of detecting the limited turnover of ion transporters of about 1 to 1000 molecules per second—about 10⁴ times less than most ion channels.

The problem arising from the state of the art therefore is to identify a robust assay with a very good sensitivity and usefulness for high throughput screening and profiling of NCX modulators that will. The solution of that problem is provided by the present invention.

### SUMMARY OF THE INVENTION

One subject-matter of the present invention refers to an assay for determining the activity of NCX protein wherein:
a) cells expressing NCX are provided;
b) a colored substance for determining intracellular calcium is provided;
c) cells are contacted with a NCX activity activator; and
d) the calcium mediated change in the luminescent signal from said colored substance is compared to a luminescent signal produced in a control experiment.

Another subject-matter of the present invention refers to an assay for determining the activity of NCX protein in response to the addition of a compound wherein:
a) cells expriming NCX are provided;
b) a colored substance for determining intracellular calcium is provided;
c) cells are contacted with a compound, wherein said cells have been treated, prior to treating with said compound, with a NCX activity activator; and
d) the calcium mediated change in the luminescent signal from said colored substance is compared to a luminescent signal produced in a control experiment.

In general, the NCX protein used was of mammalian origin, and in particular of human origin. The NCX protein is selected from NCX1, NCX2, NCX3, NCX4, NCX5, NCX6 and/or NCX7, in particular NCX1, NCX2 and/or NCX3.

In general, the cells used in the assay of the present invention can be derived from any eukaryotic organism. In a preferred embodiment, the cells are mammalian cells. In a more preferred embodiment, the cells are CHO (CCL-61), HEK (CCL-1573), COS7 (CRL-1651) and/or JURKAT (CRL-1990) cells.

In particular, the NCX activity activator used in the assay of the present invention is ionomycin.

In a preferred embodiment, said colored substance is added to the cells as a dye precursor capable of entering the cells and being hydrolyzed to a dye, whereby the dye complexes with calcium in said cells and provides a luminescent signal. Further said dye precursor can be preferably an acetoxymethylester derivate and said dye can be preferably the calcium sensitive fluorescence dye fluo-4. In a more preferred embodiment, said luminescent signal is fluorescence and said monitoring step c) employs a FLIPR device.

The invention pertains further to the use of an assay as mentioned before to test a compound for activity as an agonist or antagonist of NCX. In another preferred embodiment, the invention pertains to the use of an assay as mentioned before for the diagnosis of a disease associated with a NCX altered expression.

The invention pertains further to a kit of parts comprising:
a) lyophilized cells expriming NCX protein;
b) a colored substance;
c) a compound buffer; and
d) a colored substance buffer.

In a preferred embodiment of the kit of parts of the present invention, said colored substance is the calcium sensitive fluorescence dye fluo-4. In another preferred embodiment, the NCX protein used was of mammalian origin, and in particular of human origin. The NCX protein is selected from NCX1, NCX2, NCX3, NCX4, NCX5, NCX6 and/or NCX7, in particular NCX1, NCX2 and/or NCX3. In another preferred embodiment,

The invention pertains further to the use of a kit of parts as mentioned before to test a compound for activity as an agonist or antagonist of NCX. In another preferred embodiment, the invention pertains to the use of a kit of parts as mentioned before for the diagnosis of a disease associated with a NCX altered expression.

### DETAILED DESCRIPTION OF THE INVENTION

The term “assay” refers to a procedure where a property of a system or object is measured. Assay is a short hand commonly used term for biological assay and is a type of in vitro experiment. Assays are typically conducted to measure the effects of a substance on a living organism. Assays may be qualitative or quantitative, they are essential in the development of new drugs.

The subject assay provides a broad dynamic range so that the activity of a NCX protein can be determined. In particular the present invention makes available a rapid, effective assay for screening and profiling pharmaceutically effective compounds that specifically interact with and modulate the activity of a NCX protein.

The term “NCX protein” or “NCX” in context of the present invention shall mean any one of the list of the following Na⁺/Ca²⁺ exchanger proteins either alone or in combination with each other: NCX1, NCX2, NCX3, NCX4, NCX5, NCX6, NCX7.

Especially preferred are NCX1, NCX2 and/or NCX3 which amino acid sequences correspond,

respectively, to SEQ ID NO: 1, SEQ ID NO: 2 and SEQ ID NO: 3.

Such NCX protein could be derived from any vertebrate and in particular mammalian species (e.g. dog, horse, bovine, mouse, rat, canine, rabbit, chicken, anthropoid, human or others). The NCX could be isolated from tissue probes of such vertebrate organisms or could be manufactured by means of recombinant biological material that is able to express the NCX protein.

The term “NCX protein” refers to polypeptides, polymorphic variants, mutants, and interspecies homologues that have an amino acid sequence that has greater than about 80% amino acid sequence identity, 85%, 90%, preferably 91%, 92%, 93%, 94%, 95%, 96%, 97%, 98% or 99% or greater amino acid sequence identity, preferably over a region of at least about 25, 50, 100, 200, or 500, or more amino acids, to an amino acid sequence encoded by the nucleic acid sequence contained in SEQ ID NO: 1, SEQ ID NO: 2 and SEQ ID NO: 3.

The term “biological material” means any material containing genetic information and capable of reproducing itself or being reproduced in a biological system. Recombinant biological material is any biological material that was produced, has been changed or modified by means of recombinant techniques well known to a person skilled in the art.

The following references are examples of the cloning of particular NCX proteins: The canine Na⁺/Ca²⁺ exchanger NCX1 has been cloned by Nicoll, D A. et al. (Science. 250(4980): 562-5, 1990; Title: Molecular cloning and functional expression of the cardiac sarcolemmal Na(+)-Ca2+ exchanger.). The human Na⁺/Ca²⁺ exchanger NCX1 has been cloned by Komuro, I., et al. (Proc. Natl. Acad. Sci. U.S.A. 89 (10), 4769- 4773, 1992; Title: Molecular cloning and characterization of the human cardiac Na⁺/Ca²⁺ exchanger cDNA) and by Kofuji, P. et al. (Am. J. Physiol. 263 (Cell Physiol. 32): C1241-C1249, 1992; Title: Expression of the Na—Ca exchanger in diverse tissues: a study using the cloned human cardiac Na—Ca exchanger). The human Na⁺/Ca²⁺ exchanger NCX2 has been cloned by Li, Z. et al. (J. Biol. Chem. 269(26): 17434-9, 1994; Title: Cloning of the NCX2 isoform of the plasma membrane Na(+)-Ca2+ exchanger). The rat Na⁺/Ca²⁺ exchanger NCX3 has been cloned by Nicoll, D A. et. al. (J. Biol. Chem. 271(40): 24914-21. 1996; Title: Cloning of a third mammalian Na⁺/Ca²⁺ exchanger, NCX3). The human Na⁺/Ca²⁺ exchanger NCX3 has been cloned by Gabellini, N. et. al. (Gene. 298: 1-7, 2002; Title: The human SLC8A3 gene and the tissue-specific Na⁺/Ca²⁺ exchanger 3 isoforms).

The terms “polypeptide”, “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “activity of NCX protein” refers to the mechanism of removing intracellular Ca²⁺ from a cell. In heart, it extrudes Ca²⁺ that has entered through Ca²⁺ channels to initiate contraction, while Na⁺ enters the heart cell. Its relevance in cardiovascular diseases is e.g. illustrated in Hobai, J A & O'Rourke, B (2004) Expert Opin. Investig. Drugs, 13, 653-664. Therefore, pharmaceutical industry has developed compounds inhibiting the NCX as e.g. described in Iwamoto, T. et al. (2004) J. Biol. Chem., 279, 7544-7553. The Na⁺/Ca²⁺ exchanger electrogenically transports three to four Na⁺ for every Ca²⁺ that moves in the opposite direction as e.g. shown by electrophysiological means in Hinata, M. et al. (2002) J. Physiol. 545, 453-461. The NCX is able to maintain the cytoplasmic Ca²⁺ concentration ([Ca²⁺] in) three to four orders of magnitude below the extracellular Ca²⁺ concentration ([Ca²⁺] out). Nevertheless, the direction of net Ca²⁺ transport depends on the electrochemical gradient of Na⁺. Simultaneous and consecutive transport models have been suggested for Na⁺ and Ca²⁺ translocations, and a bulk of evidence favors the latter. The activity of NCX protein is determined by measuring the enhanced luminescence resulting from a suitable colored substance complexing with calcium.

The term “cells expressing NCX” refers to cells expressing the exchanger of interest endogenously or recombinant cells.

The term “recombinant” when used with reference, e. g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. In the present invention this typically refers to cells that have been transfected with nucleic acid sequences that encode NCX proteins.

The assay is performed simply by growing the cells in an appropriate container with a suitable culture medium. The cell may be a naturally occurring cell, a native cell, an established cell line, a commercially available cell, a genetically modified cell, etc. so long as the cell is able to be maintained during the assay and desirably growing in a culture medium.

Suitable cells for generating the subject assay include prokaryotes, yeast, or higher eukaryotic cells, especially mammalian cells. Prokaryotes include gram negative and gram positive organisms. The cells will usually be mammalian cells, such as human cells, mouse cells, rat cells, Chinese hamster cells, etc. Cells that are found to be convenient include CHO, COS7, JURKAT, HeLa, HEKs, MDCK and HEK293 cells.

Cells may be prepared with the well known methods (Current protocols in cell biology, John Wiley & Sons Inc, ISBN: 0471241059) or may be bought (Invitrogen Corp., Sigma-Aldrich Corp., Stratagene).

The term “colored substance” refers in particular to a calcium sensitive fluorescence dye. The dye precursor is characterized by not being luminescent under the conditions of the assay, being an ester capable of entering the cells and that is hydrolyzed intracellularly to the luminescent oxy compound, and providing enhanced luminescence upon complexing with calcium. The esters are chosen to be susceptible to hydrolysis by intracellular hydrolases.

The term “capable of entering the cells” means that the precursors are able to cross the cellular membrane and be hydrolyzed in the cells, the dye precursor enters the cells under specific conditions of pH, temperature, etc., enters the cells at different speeds or does not enter the cells under specific conditions.

The colored substance is added to the cells using the well known protocols (Current protocols in cell biology, John Wiley & Sons Inc, ISBN: 0471241059).

The use of a colored substance is conventional and commercially available reagents (Invitrogen Corp.) as well as reagents synthesized in laboratory can be used.

A number of commercially available dyes fulfilling the above requirements are known. Fluorescent dyes for monitoring Ca²⁺ are well known and described in detail in section 20.1-20.4 of the Molecular Probes catalog, 9th edition. They usually have two bis-carboxymethylamino groups attached to a fluorescent nucleus such as fluoresceins, rhodamines, coumarins, aminophenylindoles, and others. For the most part the compounds are 3,6-dioxy substituted xanthenes, where in the precursor the oxy groups are substituted and in the luminescent dye they are unsubstituted. Usually there are acetoxymethyl groups protecting the phenols and acids. See, for example, Fluo3/4, Fura2/3, calcein green, etc. Hydrolysis of the acetyl group results in the luminescent product. The precursors are able to cross the cellular membrane and be hydrolyzed in the cell.

The term “luminescence” refers to a “cold light”, light from other sources of energy, which can take place at normal and lower temperatures. In luminescence, some energy source kicks an electron of an atom out of its “ground” (lowest-energy) state into an “excited” (higher-energy) state; then the electron gives back the energy in the form of light so it can fall back to its “ground” state. There are several varieties of luminescence, each named according to what the source of energy is, or what the trigger for the luminescence is.

The term “fluorescence” refers to a luminescence that is mostly found as an optical phenomenon in cold bodies, in which the molecular absorption of a photon triggers the emission of another photon with a longer wavelength. The energy difference between the absorbed and emitted photons ends up as molecular vibrations or heat. Usually the absorbed photon is in the ultraviolet range, and the emitted light is in the visible range, but this depends on the absorbance curve and Stokes shift of the particular fluorophore. Fluorescence is named after the mineral fluorite, composed of calcium fluoride, which often exhibits this phenomenon.

Fluorescence from the indicator dyes can be measured with a luminometer or a fluorescence imager. One preferred detection instrument is the Fluorometric Imaging Plate Reader (FLIPR) (Molecular Devices, Sunnyvale, Calif.). The FLIPR is well suited to high throughput screening using the methods of the present invention as it incorporates integrated liquid handling capable of simultaneously pipetting to 96 or 384 wells of a microtiter plate and rapid kinetic detection using a argon laser coupled to a charge-coupled device imaging camera.

An alternative to the use of calcium indicator dyes is the use of the aequorin system. The aequorin system makes use of the protein apoaequorin, which binds to the lipophilic chromophore coelenterazine forming a combination of apoaequorin and coelenterazine that is known as aequorin. Apoaequorin has three calcium binding sites and, upon calcium binding, the apoaequorin portion of aequorin changes its conformation. This change in conformation causes coelenterazine to be oxidized into coelenteramide, CO2, and a photon of blue light (466 nm). This photon can be detected with suitable instrumentation. For reviews on the use of aequorin, see Créton et al., 1999, Microscopy Research and Technique 46:390-397; Brini et al., 1995, J. Biol. Chem. 270:9896-9903; Knight & Knight, 1995, Meth. Cell. Biol. 49:201-216. Also of interest may be U.S. Pat. No. 5,714,666 which describes methods of measuring intracellular calcium in mammalian cells by the addition of coelenterazine co-factors to mammalian cells that express apoaequorin.

“Inhibitors”, “activators”, and “modulators” of NCX polynucleotide and polypeptide sequences are used to refer to activating, inhibitory, or modulating molecules identified using cell-based assays of NCX polynucleotide and polypeptide sequences.

“Inhibitors” are compounds that, e. g., bind to, partially or totally block activity, decrease, prevent, delay activation, inactivate, desensitize, or down regulate the activity or expression of NCX proteins, e. g., antagonists.

“Activators” are compounds that increase, open, activate, facilitate, enhance activation, sensitize, agonize, or up regulate NCX protein activity. A preferred NCX activator is ionomycin, an ionophore that comes from Streptomyces conglobatus.

Inhibitors, activators, or modulators also include genetically modified versions of NCX proteins, e. g., versions with altered activity, as well as naturally occurring and synthetic ligands, antagonists, agonists, peptides, cyclic peptides, nucleic acids, antibodies, antisense molecules, ribozymes, small organic molecules and the like.

The term “compound” or “test compound” or “test candidate” or grammatical equivalents thereof describes any molecule, either naturally occurring or synthetic, e. g., protein, oligopeptide, small organic molecule, polysaccharide, lipid, fatty acid, polynucleotide, oligonucleotide, etc., to be tested for the capacity to modulate NCX activity (Current protocols in molecular biology, John Wiley & Sons Inc, ISBN: 0471250961). The test compound can be in the form of a library of test compounds, such as a combinatorial or randomized library that provides a sufficient range of diversity (Current protocols in molecular biology, John Wiley & Sons Inc, ISBN: 0471250937). Test compounds are optionally linked to a fusion partner, e. g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e. g., enhancing activity, creating variants of the lead compound, and evaluating the property and activity of those variant compounds. Preferably, high throughput screening (HTS) methods are employed for such an analysis.

Said inhibitor, activator and test compound may be added to the cells by injection into the culture medium after the cells have grown or they may be present in the culture medium prior to the cell growth (Current protocols in cell biology, John Wiley & Sons Inc, ISBN: 0471241059).

The cells may be grown to the appropriate number on the inhibitor, activator and/or test compound or they may be placed on it and used without further growth. The cells may be attached to the inhibitor, activator and/or test compound or, in those embodiments where the cells are placed or grown in wells, the cells may be suspension cells that are suspended in the fluid in the wells.

The term “control experiment” refers to different kinds of experiments that should be run together. The skilled person will recognize that it is generally beneficial to run controls together with the methods described herein.

For example, it will usually be helpful to have a control for the assay for determining the activity of NCX protein in which the cells are preferably essentially identical to the cells that are used in the assay except that these cells would not express the NCX protein of interest. Furthermore, it will usually be helpful to have a control for the assay for determining the activity of NCX protein in response to the addition of a compound in which the compounds are tested in the assay of the invention against cells that preferably are essentially identical to the cells that are used in the assay except that these cells would not express the NCX protein of interest. In this way it can be determined that compounds which are identified by the assay are really exerting their effects through the NCX protein of interest rather than through some unexpected non-specific mechanism. One possibility for such control cells would be to use non-recombinant parent cells where the cells of the actual experiment express the NCX protein of interest.

Other controls for the assay for determining the activity of NCX protein in response to the addition of a compound would be to run the assay without adding a test compound (low control) and to run the assay with a high concentration of test compound (high control). Other types of controls would involve taking compounds that are identified by the assay of the present invention as agonists or antagonists of NCX proteins of interest and testing those compounds in the methods of the prior art in order to confirm that those compounds are also agonists or antagonists when tested in those prior art methods.

Furthermore, one skilled in the art would know that it also desirable to run statistical analysis by comparing the assay values to standard values.

The terms “agonist” and “antagonist” refer to receptor effector molecules that modulate signal transduction via a receptor. Receptor effector molecules are capable of binding to the receptor, though not necessarily at the binding site of the natural ligand. Receptor effectors can modulate signal transduction when used alone, i.e. can be surrogate ligands, or can alter signal transduction in the presence of the natural ligand, either to enhance or inhibit signaling by the natural ligand. For example, “antagonists” are molecules that block or decrease the signal transduction activity of receptor, e.g., they can competitively, noncompetitively, and/or allosterically inhibit signal transduction from the receptor, whereas “agonists” potentiate, induce or otherwise enhance the signal transduction activity of a receptor.

The term “disease associated with a NCX altered expression” refers to dilated cardiomyopathy, coronary heart disease, arrhythmia, heart failure, etc.

For convenience, the colored substance and other components of the assay may be provided in kits, where the colored substance may be present as a reconstitutable powder or as a cooled solution on ice, in a buffer. The kit may also include buffer, activator, inhibitor, test compound, cells expriming NCX protein, etc. Cells may be present as lyoplilized cells. Said kit of parts can be used as a diagnostic kit for diagnosing dilated cardiomyopathy, coronary heart disease, arrhythmia, heart failure, etc.

The following figures and examples shall describe the invention in further details, describing the typical results of the fluorescence based cellular NCX assay, without limiting the scope of protection.

Exemplification

1. Assay Procedure

1.1. Assay Reagents

The following chemical compositions are used as reagents for the assay:

| Reagent | Chemicals | Remarks |
|---|---|---|
| Assay buffer | 3.5 mM CaCl₂ | Probenecid is added on the |
| | 133.8 mM NaCl | day of use from a freshly |
| | 4.7 mM KCl | prepared 1 M solution in |
| | 1.25 mM MgCl₂ | 1 N NaOH. |
| | 0.01% Pluronic F-127 | |
| | 10 mM Hepes/NaOH | |
| | pH 7.5 | |
| | 5 mM Glucose | |
| | 2.5 mM Probenecid | |
| Dye loading | Assay buffer containing | Fluo-4/AM is added from a |
| buffer | 2 μM Fluo-4/AM | 1 mM stock solution in |
| | 0.1% BSA | DMSO |
| Compound buffer | Assay buffer | Compounds are added |
| | Various compound | from a 10 mM |
| | concentrations | stock solution in |
| | | DMSO |
| Ionophor | Assay buffer containing | Ionomycin is added |
| solution | 0.3% BSA | from a 10 mM |
| | 6 μM Ionomycin | stock solution in |
| | | DMSO |
| Positive control | low) Ionophor solution | A000135933 is added |
| buffers | high) Assay buffer | from a 10 mM |
| | 15-45 μM A000135933 | stock solution in |
| | | DMSO |

1.2. Assay Procedure
1] 20-24 h before the experiment, cells are suspended in growth medium (Nutrient Mixture F12 (HAM) Invitrogen, Karlsruhe, 5% FCS, Biochrom, Berlin) without antibiotics and seeded into 96-well black clear bottom plates (25000 cells/well in 100 μl).
2] Medium is discarded and subsequently 100 μl of dye loading buffer are added and plates are incubated dark for 75 min at RT.
3] Dye loading buffer is removed by washing three times with 100 μl assay buffer. Buffer is discarded
4] 80 μl from compound plates are added and plates are stored for 30 min at 16° C.
5] Plates are transferred into the FLIPR and assayed using the following protocol (including 40 μl addition from ionophor plate):

1.1 FLIPR Experimental Setup Parameters

| Exposure | 0.5 sec (at 1.2 W) |
|---|---|
| F-Stop | F/2 |
| Filter | 1 |

1.1.1 Graph Setup

| | |
|---|---|
| Subtract Bias Based on Sample: | off |
| Spatial Uniformity Correction: | off |
| Negative Control Correction: | off |

1.1.2 First Sequence

| | |
|---|---|
| Initial Period | 2 sec |
| Initial Count | 100 frames |
| Add After Frame | 5 |
| Add Height | 70 μl |
| Add Speed | 40 μl/sec |
| Add Volume | 40 μl |
| Mix | 1 × 40 μl |

Statistics

| | |
|---|---|
| Statistic 1 | sum 25-45 (bias off) |

1.3. Data Analysis

Inhibitory Activity of Test Compounds in NCX Cells:

Calculation of Inhibition:

Calculations are based on the statistics export. Raw data are converted to inhibition according to:

$$\% - INHIBITION = 100 \times ( \frac{sample - mean low control}{mean high control - mean low control} )$$

Mean high control is derived from the average difference of eight paired samples of 10 or 30 μM A000135933 with ionomycine. Mean low control is derived from ionomycine controls. Compounds which increases the basal fluorescence higher than 1.3 fold are discarded.

2. Assay examples

2.1. Response of the High and Low Controls.

The typical fluorescence response of the high and low controls after addition of 2 μM lonomycine is shown in FIG. 2 and is as following: If the NCX1 is active (low control) calcium entering the cells after lonomycine addition is transported out of the cells. After a few seconds the initial calcium load of the cells is reestablished. Inhibition of NCX1 leads to a fluorescence increase after lonomycine addition due to an increase of cytosolic calcium (high control, 30 μM A000135933).

2.2. Tool Substance: A000135933

The new NCX1 inhibitor A000135933 was found in the first HTS screen. FIGS. 3, 4 and 5 show a typical dose dependent response of different concentrations of A000135933. A000135933 was a good NCX1 Inhibitor with a mean IC₅₀ of 5.9 μM and since that time used as tool substance in the assays. An IC₅₀ of this compound is added on every plate as control. The S/B ratio and the z′ value for this example were very good. Together with the IC₅₀ of A000135933 these parameters were used to indicate good assay performance for every plate:

1. S/B greater than two.

2. z′ value between 0.5 and 0.7.

3. IC₅₀ of the tool compound A000135933 has to be around the mean of 5.9 μM.

2.3. Tool Substance: Assay Example

An assay was performed with four compounds IC₅₀s in duplicate (FIG. 6). The four compounds are from the same compound class. One compound was a good NCX1 inhibitor (A000135933), two compounds show moderate inhibition (A000136648, A000104243) and one was not active in the concentration range (A000103746). This example indicates that the assay is suitable so screen NCX1 inhibitors and to establish structure activity relationships.

2.4. Correlation with Electrophysiology

The comparison of the data derived from the fluorescence-based assay with a direct electrophysiology method (longate's SURFE²R technology) is the best way to estimate the performance of this assay. The correlation of these two very different techniques is quite good (FIG. 7).

The Inhibition measured with the SURFE²R was higher (mean 14%) except for one compound than the inhibition derived from the indirect FLIPR assay.

### DESCRIPTION OF THE FIGURES

FIG. 1:

FIG. 1a shows the polynucleotide sequence of NCX1 represented by SEQ ID NO: 1.

FIG. 1b shows the polynucleotide sequence of NCX2 represented by SEQ ID NO: 2.

FIG. 1c shows the polynucleotide sequence of NCX3 represented by SEQ ID NO: 3.

FIG. 2:

Fluorescence signal of the CHO-NCX1 cells after lonomycine addition. Inhibition of NCX1 (high control, 30 μM A000135933, red) leads to a fluorescence increase due to an increase of cytosolic calcium. Active NCX1 establish the initial calcium load after a few seconds (low control, black).

FIG. 3:

Raw data: Kinetic of the fluorescence changes after ionomycine addition for different concentrations of A000135933. The sum of the fluorescence values from 50 to 90s were used to calculate the percentage fluorescence changes in comparison to the controls. The results are shown in FIG. 4.

FIG. 4:

Assay statistic for a 96 well plate with high and low controls and different concentrations of A000135933. Calculated signal to background ratio (S/B), z′ and increase of the fluorescence between 50 and 90 seconds of different concentrations of A000135933 are listed (s.a. FIG. 2). For this example the calculated IC₅₀ of A000135933 was 7.16 μM (mean IC₅₀: 5.9 μM).

FIG. 5:

Illustration of the percentage fluorescence increase in comparison to the compound concentration of A000135933 and the corresponding fit curve. For this example the calculated IC₅₀ of A000135933 was 7.16 μM (mean IC₅₀: 5.9 μM).

FIG. 6:

FIG. 6 shows the raw data print out from the FLIPR.

FIG. 7:

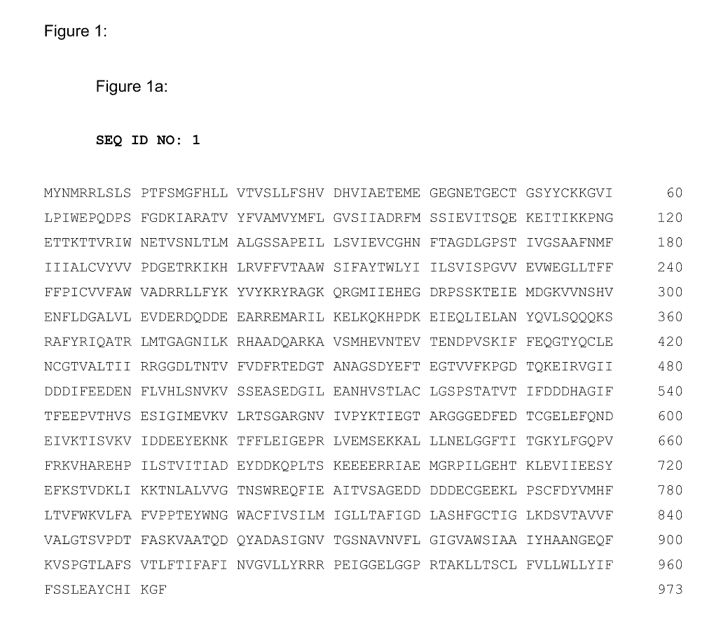

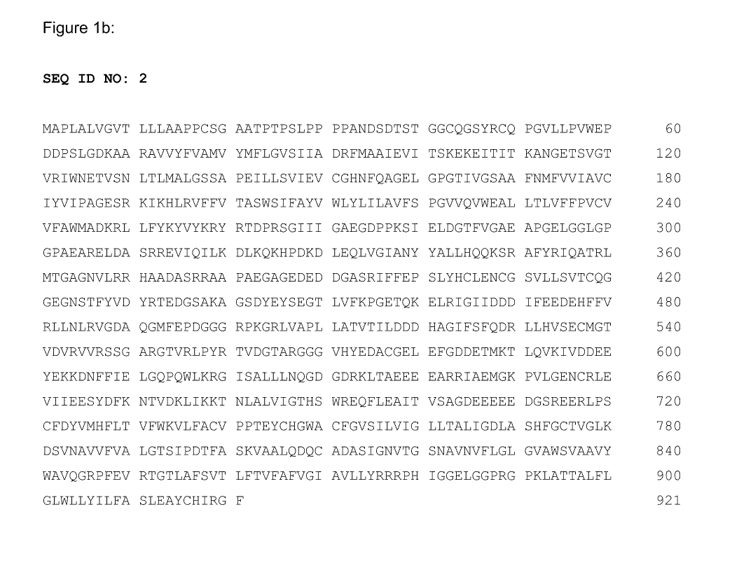

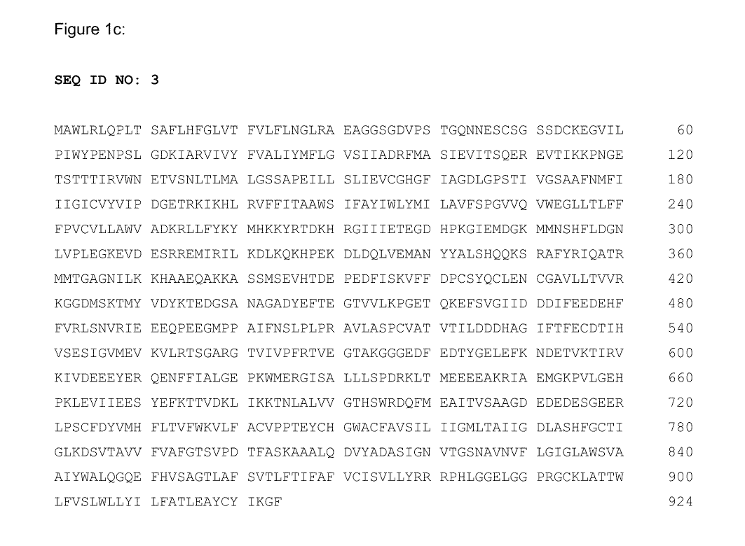

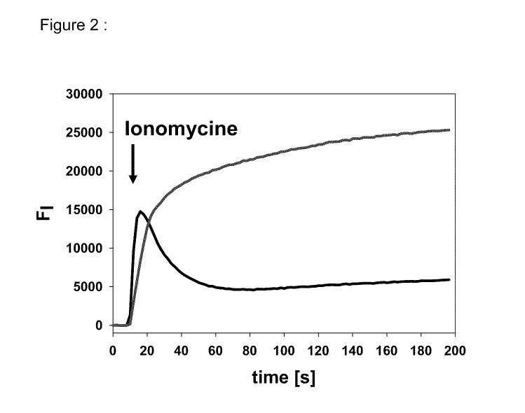

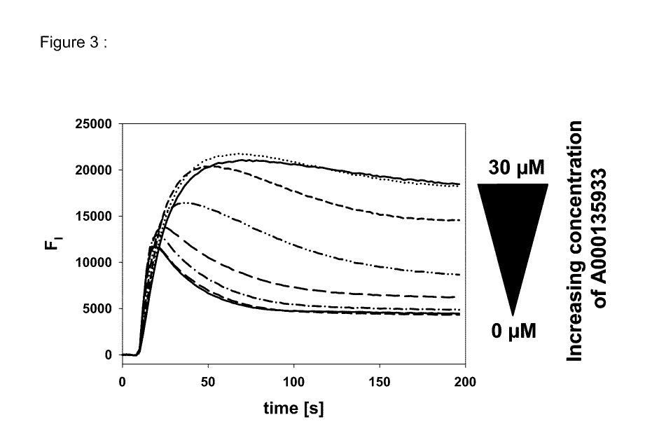

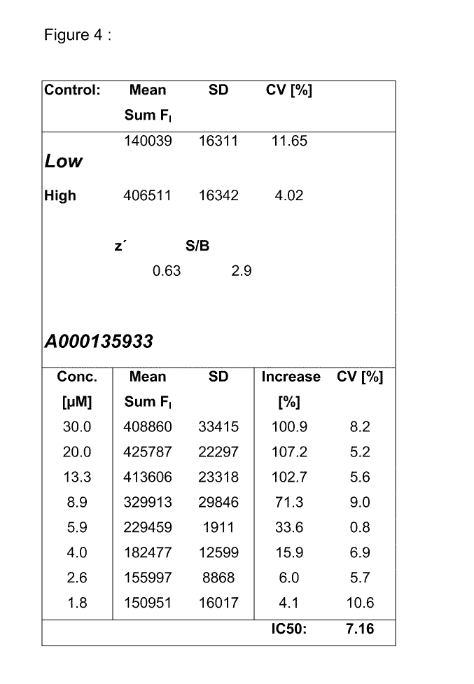

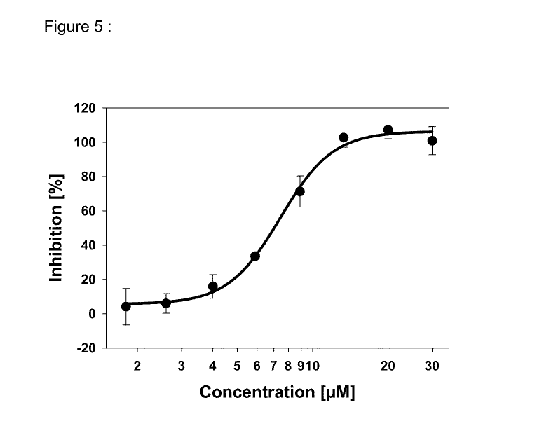

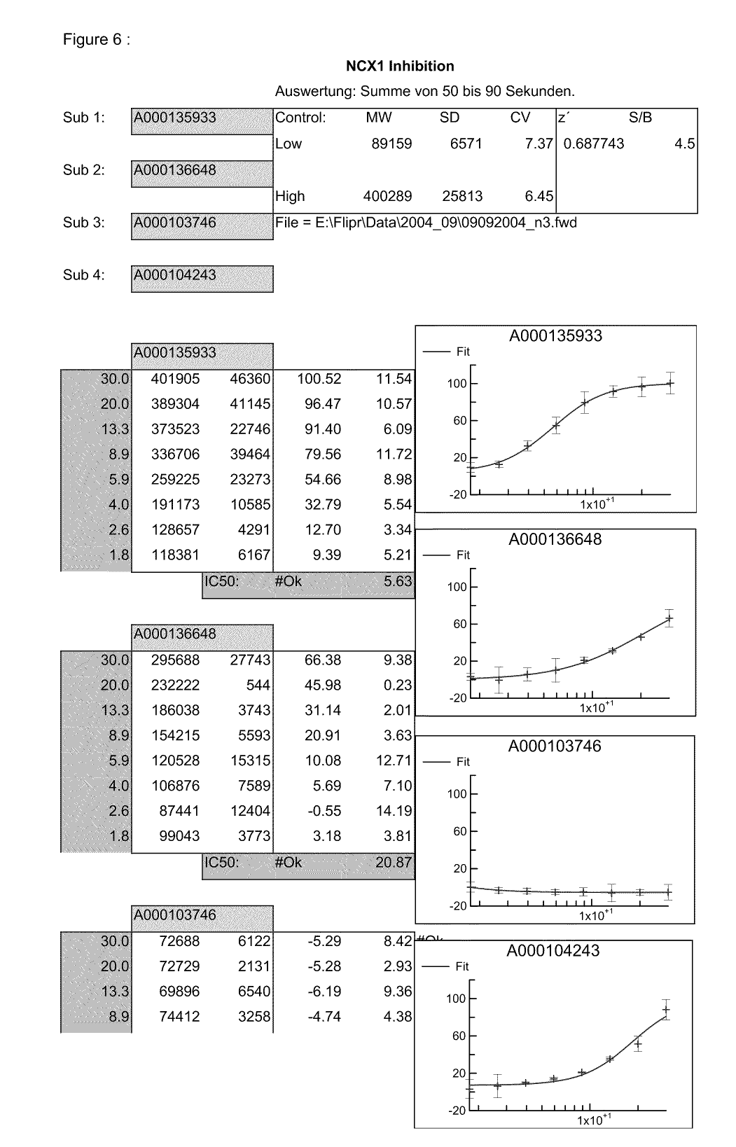

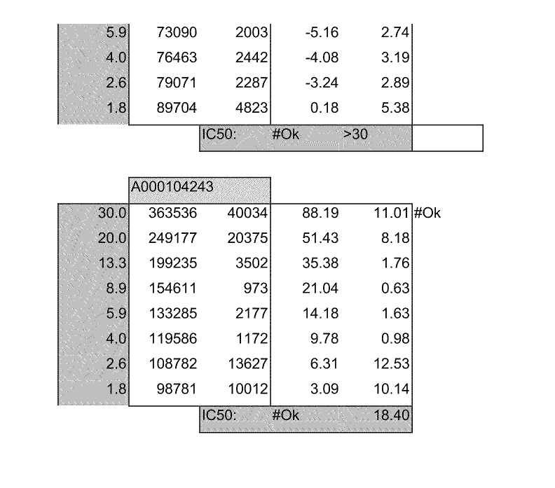

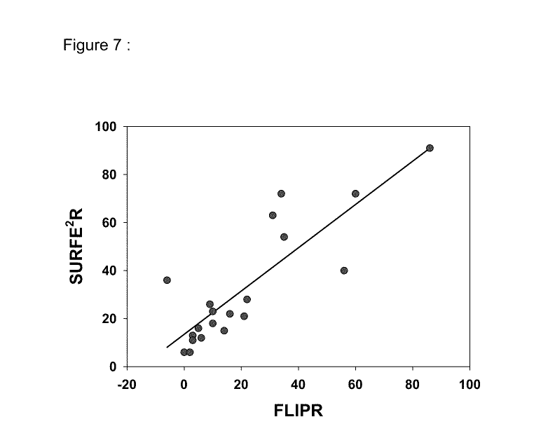

Correlation between the NCX1 fluorescence based FLIPR assay with the electrophysiology based SURFE²R technology of one compound class. The inhibition of NCX1 was measured in both cases at 10 μM.

1. An assay for determining the activity of NCX protein, said assay comprising:
a) providing cells expressing NCX;
b) providing a luminescent colored substance for determining intracellular calcium;
c) contacting cells with a NCX activity activator; and
d) comparing the calcium mediated change in the luminescent signal from said colored substance to a luminescent signal produced in a control experiment.

2. The assay according to claim 1, wherein the NCX protein is a NCX protein selected from the group consisting of NCX1, NCX2 and NCX3.

3. The assay according to claim 1, wherein the NCX protein is of mammalian origin.

4. The assay according to claim 1, wherein the cells are selected from the group consisting of: CHO, HEK, COS7 and JURKAT cells.

5. The assay according to claim 1, wherein said colored substance is added to the cells as a dye precursor capable of entering the cells and being hydrolyzed to a dye, whereby the dye complexes with calcium in said cells and provides a luminescent signal.

6. The assay according to claim 1, wherein said luminescent signal is fluorescence and said assay employs a FLIPR device.

7. The assay according to claim 5, wherein said dye precursor is an acetoxymethylester derivate.

8. The assay according to claim 5, wherein said dye is the calcium sensitive fluorescence dye fluo-4.

9. The assay according to claim 1, wherein said NCX activity activator is ionomycin.

10. The assay according to claim 1 further comprising providing a compound to be tested for activity as an agonist or antagonist of NCX.

11. The assay according to claim 1 wherein said comparing facilitates diagnosis of a disease associated with a NCX altered expression.

12. An assay for determining activity of NCX protein in response to the addition of a compound, said assay comprising:
a) providing cells expriming NCX;
b) providing a luminescent colored substance for determining intracellular calcium;
c) contacting cells with a compound, wherein said cells have been treated, prior to treating with said compound, with a NCX activity activator; and
d) comparing the calcium mediated change in the luminescent signal from said colored substance to a luminescent signal produced in a control experiment.

13. An assay according to claim 12, wherein the NCX protein is a NCX protein selected from the group consisting of NCX1, NCX2 and NCX3.

14. The assay according to claim 12, wherein the NCX protein is of mammalian origin.

15. The assay according to claim 12, wherein the cells are selected from the group consisting of: CHO, HEK, COS7 and JURKAT cells.

16. The assay according to claim 12, wherein said colored substance is added to the cells as a dye precursor capable of entering the cells and being hydrolyzed to a dye, whereby the dye complexes with calcium in said cells and provides a luminescent signal.

17. The assay according to claim 12, wherein said luminescent signal is fluorescence and said assay employs a FLIPR device.

18. The assay according to claim 16, wherein said dye precursor is an acetoxymethylester derivate.

19. The assay according to claim 16, wherein said dye is the calcium sensitive fluorescence dye fluo-4.

20. The assay according to claims 12, wherein said compound is a NCX antagonist.

21. The assay according to claim 12, wherein said NCX activator is ionomycin.

22. A kit comprising:
a) lyophilized cells expriming NCX protein;
b) a colored substance;
c) a compound buffer; and
d) a colored substance buffer.

23. The kit according to claim 22, wherein said colored substance is the calcium sensitive fluorescence dye fluo-4.

24. The kit according to claim 22, wherein the NCX protein is a NCX protein selected from the group consisting of NCX1, NCX2 and NCX3.

25. The kit according to claims 22, wherein the NCX protein is of mammalian origin.

26-27. (canceled)

28. The assay according to claim 3, wherein the NCX protein is selected from the group consisting of rat, mouse, dog, bovine, pig, ape and human NCX protein.

29. The assay according to claim 14, wherein the NCX protein is selected from the group consisting of rat, mouse, dog, bovine, pig, ape and human NCX protein.

30. The kit according to claim 25, wherein the NCX protein is selected from the group consisting of rat, mouse, dog, bovine, pig, ape and human NCX protein.


SEQUENCE LISTING (3 sequences)

SEQ ID NO: 1 - protein, 973 aa, Homo sapiens
MYNMRRLSLSPTFSMGFHLLVTVSLLFSHVDHVIAETEMEGEGNETGECTGSYYCKKGVILPIWEPQDPSFGDKIARATVYFVAMVYMFLGVSIIADRFMSSIEVITSQEKEITIKKPNGETTKTTVRIWNETVSNLTLMALGSSAPEILLSVIEVCGHNFTAGDLGPSTIVGSAAFNMFIIIALCVYVVPDGETRKIKHLRVFFVTAAWSIFAYTWLYIILSVISPGVVEVWEGLLTFFFFPICVVFAWVADRRLLFYKYVYKRYRAGKQRGMIIEHEGDRPSSKTEIEMDGKVVNSHVENFLDGALVLEVDERDQDDEEARREMARILKELKQKHPDKEIEQLIELANYQVLSQQQKSRAFYRIQATRLMTGAGNILKRHAADQARKAVSMHEVNTEVTENDPVSKIFFEQGTYQCLENCGTVALTIIRRGGDLTNTVFVDFRTEDGTANAGSDYEFTEGTVVFKPGDTQKEIRVGIIDDDIFEEDENFLVHLSNVKVSSEASEDGILEANHVSTLACLGSPSTATVTIFDDDHAGIFTFEEPVTHVSESIGIMEVKVLRTSGARGNVIVPYKTIEGTARGGGEDFEDTCGELEFQNDEIVKTISVKVIDDEEYEKNKTFFLEIGEPRLVEMSEKKALLLNELGGFTITGKYLFGQPVFRKVHAREHPILSTVITIADEYDDKQPLTSKEEEERRIAEMGRPILGEHTKLEVIIEESYEFKSTVDKLIKKTNLALVVGTNSWREQFIEAITVSAGEDDDDDECGEEKLPSCFDYVMHFLTVFWKVLFAFVPPTEYWNGWACFIVSILMIGLLTAFIGDLASHFGCTIGLKDSVTAVVFVALGTSVPDTFASKVAATQDQYADASIGNVTGSNAVNVFLGIGVAWSIAAIYHAANGEQFKVSPGTLAFSVTLFTIFAFINVGVLLYRRRPEIGGELGGPRTAKLLTSCLFVLLWLLYIFFSSLEAYCHIKGF

SEQ ID NO: 2 - protein, 921 aa, Homo sapiens
MAPLALVGVTLLLAAPPCSGAATPTPSLPPPPANDSDTSTGGCQGSYRCQPGVLLPVWEPDDPSLGDKAARAVVYFVAMVYMFLGVSIIADRFMAAIEVITSKEKEITITKANGETSVGTVRIWNETVSNLTLMALGSSAPEILLSVIEVCGHNFQAGELGPGTIVGSAAFNMFVVIAVCIYVIPAGESRKIKHLRVFFVTASWSIFAYVWLYLILAVFSPGVVQVWEALLTLVFFPVCVVFAWMADKRLLFYKYVYKRYRTDPRSGIIIGAEGDPPKSIELDGTFVGAEAPGELGGLGPGPAEARELDASRREVIQILKDLKQKHPDKDLEQLVGIANYYALLHQQKSRAFYRIQATRLMTGAGNVLRRHAADASRRAAPAEGAGEDEDDGASRIFFEPSLYHCLENCGSVLLSVTCQGGEGNSTFYVDYRTEDGSAKAGSDYEYSEGTLVFKPGETQKELRIGIIDDDIFEEDEHFFVRLLNLRVGDAQGMFEPDGGGRPKGRLVAPLLATVTILDDDHAGIFSFQDRLLHVSECMGTVDVRVVRSSGARGTVRLPYRTVDGTARGGGVHYEDACGELEFGDDETMKTLQVKIVDDEEYEKKDNFFIELGQPQWLKRGISALLLNQGDGDRKLTAEEEEARRIAEMGKPVLGENCRLEVIIEESYDFKNTVDKLIKKTNLALVIGTHSWREQFLEAITVSAGDEEEEEDGSREERLPSCFDYVMHFLTVFWKVLFACVPPTEYCHGWACFGVSILVIGLLTALIGDLASHFGCTVGLKDSVNAVVFVALGTSIPDTFASKVAALQDQCADASIGNVTGSNAVNVFLGLGVAWSVAAVYWAVQGRPFEVRTGTLAFSVTLFTVFAFVGIAVLLYRRRPHIGGELGGPRGPKLATTALFLGLWLLYILFASLEAYCHIRGF

SEQ ID NO: 3 - protein, 924 aa, Homo sapiens
MAWLRLQPLTSAFLHFGLVTFVLFLNGLRAEAGGSGDVPSTGQNNESCSGSSDCKEGVILPIWYPENPSLGDKIARVIVYFVALIYMFLGVSIIADRFMASIEVITSQEREVTIKKPNGETSTTTIRVWNETVSNLTLMALGSSAPEILLSLIEVCGHGFIAGDLGPSTIVGSAAFNMFIIIGICVYVIPDGETRKIKHLRVFFITAAWSIFAYIWLYMILAVFSPGVVQVWEGLLTLFFFPVCVLLAWVADKRLLFYKYMHKKYRTDKHRGIIIETEGDHPKGIEMDGKMMNSHFLDGNLVPLEGKEVDESRREMIRILKDLKQKHPEKDLDQLVEMANYYALSHQQKSRAFYRIQATRMMTGAGNILKKHAAEQAKKASSMSEVHTDEPEDFISKVFFDPCSYQCLENCGAVLLTVVRKGGDMSKTMYVDYKTEDGSANAGADYEFTEGTVVLKPGETQKEFSVGIIDDDIFEEDEHFFVRLSNVRIEEEQPEEGMPPAIFNSLPLPRAVLASPCVATVTILDDDHAGIFTFECDTIHVSESIGVMEVKVLRTSGARGTVIVPFRTVEGTAKGGGEDFEDTYGELEFKNDETVKTIRVKIVDEEEYERQENFFIALGEPKWMERGISALLLSPDRKLTMEEEEAKRIAEMGKPVLGEHPKLEVIIEESYEFKTTVDKLIKKTNLALVVGTHSWRDQFMEAITVSAAGDEDEDESGEERLPSCFDYVMHFLTVFWKVLFACVPPTEYCHGWACFAVSILIIGMLTAIIGDLASHFGCTIGLKDSVTAVVFVAFGTSVPDTFASKAAALQDVYADASIGNVTGSNAVNVFLGIGLAWSVAAIYWALQGQEFHVSAGTLAFSVTLFTIFAFVCISVLLYRRRPHLGGELGGPRGCKLATTWLFVSLWLLYILFATLEAYCYIKGF